(19) United States
(12) Patent Application Publication
(10) Pub. No.: US 2009/0259138 A1
(43) Pub. Date: Oct. 15, 2009

(54) AUTOMATIC BIO-SIGNAL SUPERVISING SYSTEM FOR MEDICAL CARE

(76) Inventors: Chin-Teng LIN, Hsinchu County (TW); Jen-Feng CHUNG, Hsinchu County (TW); Li-Wei KO, Hsinchu City (TW); I-Ling TSAI, Yunlin County (TW); Che-Jui CHANG, Chiayi City (TW); Chih-Feng CHAO, Taichung County (TW)

Correspondence Address: Muncy, Geissler, Olds & Lowe, PLLC, P.O. BOX 1364, FAIRFAX, VA, 22038-1364, US

(21) Appl. No.: 12/424,325

(22) Filed: Apr. 15, 2009

(30) Foreign Application Priority Data
Apr. 15, 2008 (TW) 97113610

(51) Int. Cl. A61B 5/0482; A61B 5/00; A61B 5/0488

(52) U.S. Cl. 600/545; 600/300; 600/546

(57) ABSTRACT

The present invention discloses an automatic bio-signal supervising system for medical care. The automatic bio-signal supervising system utilizes a head-mounted bio-signal acquisition device, wore upon the user's head, to acquire the bio-signals, convert them into the digital bio-signals, and transmit the digital bio-signals to a signal analysis processor to perform analysis process. And the automatic bio-signal supervising system delivers a corresponding control signal to the environment control equipment based on the result of analysis, and control the environment condition to achieve the remote supervising and medical care purpose. Furthermore, the signal analysis processor in the present invention further provides a real-time warning signal or a health index, which supervise the personal biological information to avoid the unpredictable situation.

### BACKGROUND OF THE INVENTION

1. Field of the Invention

The present invention relates to a bio-signal supervising system, more particularly to an automatic bio-signal supervising system for medical care.

2. Description of the Related Art

Along with the vigorous development of electrical devices and the considerable progress of bio-technology, various health supervising systems have been developed by the researchers to reduce the cost of medical care. For example, a R.O.C. patent (No. I287976) disclosed a remote bio-signal supervising system for long-term remotely monitoring the bio-signals of the chronic patients. The above-mentioned patent utilized a bio-sensor to detect the patient's physiological signals, and utilized a distributed data server to calculate, read and store the data. Moreover, this prior are further included an incident warning transmitter to control the sending message. Here, since the bio-sensor, the distributed data server, and the incident biofeedback warning system transmitted the warning signals by the GSM module, the remote bio-signal supervising system in this prior art could provide real-time information of patients to the hospital or local on-line doctors. That is, the patients no longer need to stay in the hospital for a long time examining or supervising. However, the prior art only provided the function of supervising and warning. It did not reduce the requirement of medical care.

### SUMMARY OF THE INVENTION

One of the many objectives of the claimed invention is to provide an automatic bio-signal supervising system for medical care, which is consisted of the integration of a bio-signal acquisition system, a signal processing unit, and an environment controller, so as to reduce the system loading of the medical personnel devices and increase the efficiency of medical care devices.

Another objective of the claimed invention is to provide an automatic bio-signal supervising system for medical care for providing an active warning and health index function, so as to establish the emergence channel and assure the patient's safety.

According to the claimed invention, an automatic bio-signal supervising system for medical care is disclosed. The automatic bio-signal supervising system in the present invention collects the users' bio-signals by the head-mounted design such as a sport headband. The automatic bio-signal supervising system transmits the bio-signals by the wireless transmission protocols such as WiFi, WiMax, Zigbee, RF, optical coupler, Bluetooth, or GPRS to the backend real-time signal processing unit, and delivers a control signal based on the analysis results to control the environment equipments. The environment equipments include the warning devices, the air-conditioner, or the electrical device. The present invention connects the hospital or medical care into the dwelling house environment to achieve the remote care purpose.

According to the claimed invention, the automatic bio-signal supervising system for medical care performs same simple medical test, such as Electro-encephalogram (EEG), Electro-oculography (EOG), or Electro-myogram (EMG) test, by the elders to real-time supervise the their health condition.

Below, the embodiments of the present invention are described in detail in cooperation with the attached drawings to make easily understood the objectives, technical contents, characteristics and accomplishments of the present invention.

### BRIEF DESCRIPTION OF THE DRAWINGS

FIG. 1 is a block diagram of an automatic bio-signal supervising system according to an embodiment of the present invention.

FIG. 2A to 2C are diagrams schematically showing the usage of an embodiment of the automatic bio-signal supervising system

FIG. 3 is a flowchart showing an embodiment of operations of the automatic bio-signal supervising system.

### DETAILED DESCRIPTION OF THE INVENTION

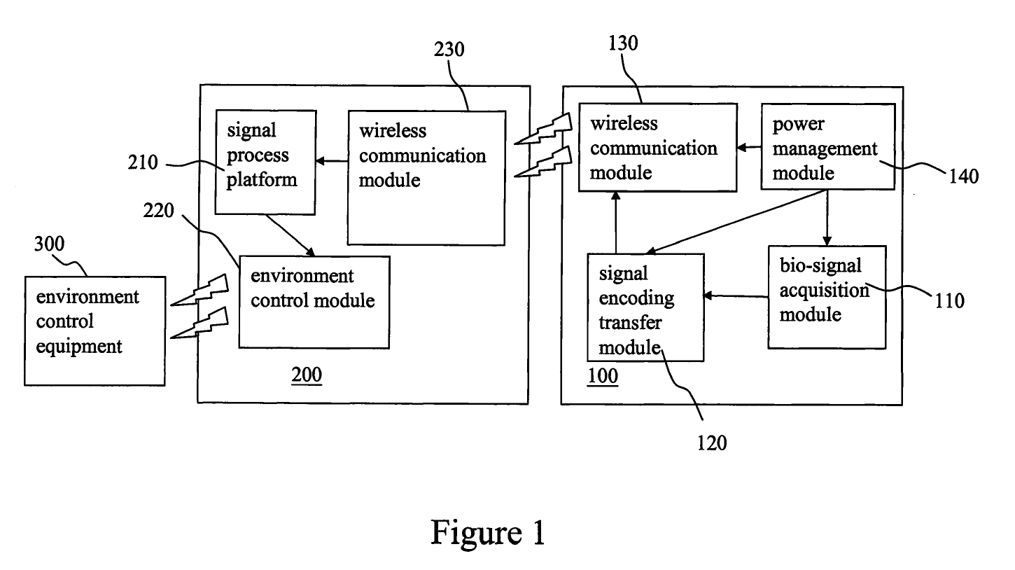

FIG. 1 is a block diagram of an automatic bio-signal supervising system according to an embodiment of the present invention. The automatic bio-signal supervising system in the present invention is basically composed of a head-mounted bio-signal acquisition device 100 and a signal analysis processor 200.

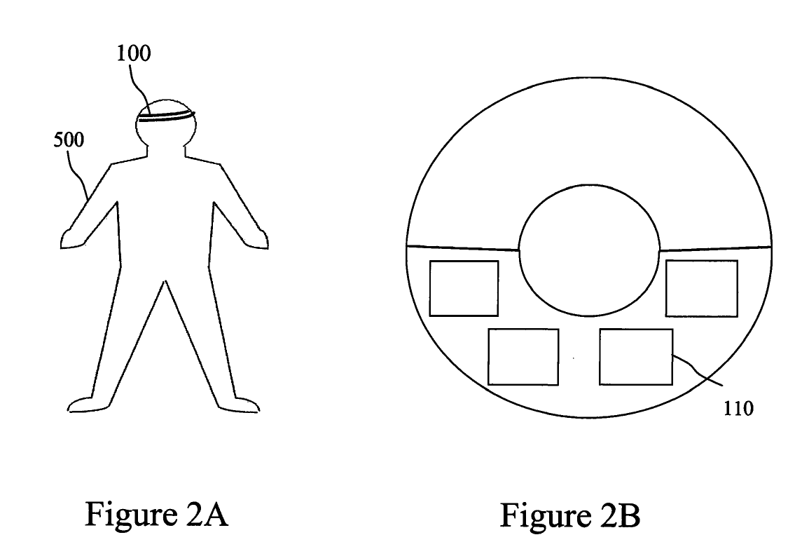

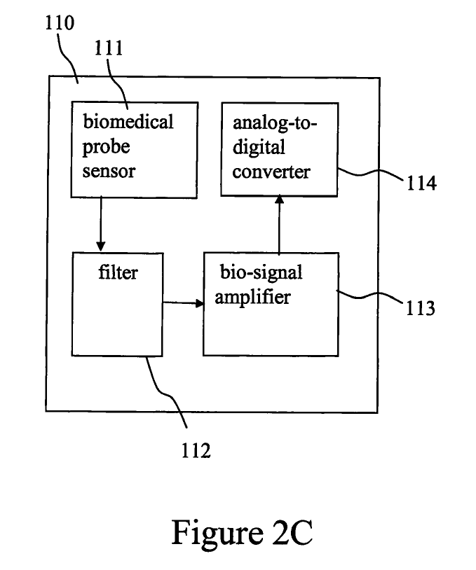

Here, the head-mounted bio-signal acquisition device 100 further includes a bio-signal acquisition module 110, a signal encoding transfer module 120, a wireless communication module 130, and a power management module 140. Please note that, the head-mounted bio-signal acquisition device 100 in this embodiment of the present invention is designed for head-wearing purpose and thereby utilizes a long stripe to tie up the patient's head, as shown in FIG. 2A˜FIG. 2C. However, as will be apparent to a personal of ordinary skill after reading this description, other embodiments of the present disclosure are also possible. For example, the bio-signal acquisition device could be an arm wrapping or leg wrapping device based on the design requirement. Considering the comfort zone of the user 500 and the weight deployment, the present embodiment utilizes the distributed circuit structure. Here, each acquisition circuit and the biomedical probe sensor 111 may also be incorporated into the bio-signal acquisition module 110 in light of changing devices easily. Each bio-signal acquisition module 110 represents a channel, and is distributed in the front of the head-mounted bio-signal acquisition device 100. When the user 500 wears the head-mounted bio-signal acquisition device 100, the biomedical probe sensor 111 in the head-mounted bio-signal acquisition device 100 will surround and attach the head of the user 100 to acquire the bio-signal directly.

The bio-signal acquisition module 110 further includes a filter 112, a bio-signal amplifier 113, and an analog-to-digital converter (ADC) 114. Because each bio-signal acquired from the forehead area has different gain and bandwidth, the embodiment of the present invention designs the required parameters based on different range for different bio-signal, as shown in the following Chart 1.

CHART 1

| Bio-signal | Bandwidth | |
|---|---|---|
| Electro-encephalogram (EEG) | 0.1~100 | Hz |
| Electro-oculography (EOG) | 0~100 | Hz |
| Electro-myogram (EMG) | 20~1 | kHz |

The filter 112 is utilized for filtering the noise of bio-signals. In the present embodiment, the bio-signal, which acquired by the biomedical probe sensor 111, may contain some noise signals, such as 60/120 Hz ground noise. The filter 112 is regulated by the design requirement and different acquired bio-signals to filter out the noise based upon the above-mentioned Chart 1.

(a) The bio-signal amplifier 113: Usually the bio-signals acquired from the biomedical probe sensor 111 are pretty slight (couple μV). Thus, the bio-signal amplifier 113 is utilized to amplify the signal properly for the following analog-to-digital transfer process.

(b) The analog-to-digital converter (ADC) 114: The bio-signals slight from the biomedical probe sensor 111 are analog signals, which indicate that they are not convenient for storing and analyzing purpose. The analog-to-digital converter (ADC) 114 in the present invention thereby is utilized for converting the analog signals into the digital signals. Moreover, the resolution of the analog-to-digital converter (ADC) 114 is a key issue as well. In this embodiment, the analog-to-digital converter (ADC) 114 is utilized resolution rate from 8 to 22 bits for normal usage. Generally, higher bit resolution indicates better accurate rate.

Additionally, the signal encoding transfer module 120 is composed of a microcontroller or a microprocessor. The signal encoding transfer module 120 is utilized for receiving the digital bio-signal from the bio-signal acquisition module 110 through a serial device, and encoding the signals before transmitting so as to reduce the baud rate and save the power. The encoding process of the signal encoding transfer module 120 includes baud rate setup (baud rate auto-adjustment), communication module initialization, communication protocol setup, and data encoding.

Next, after encoding by the signal encoding transfer module 120, the digital bio-signal is wireless transmitted to the signal analysis processor 200 by the communication protocol, such as WiFi, WiMax, Zigbee, RF, optical coupler, Bluetooth, or GPRS. That is, the present invention is embodied by any well-known communication protocols, and is not meant to be taken as limitations.

The power management module 140 is utilized for providing the required power of the head-mounted bio-signal acquisition device 100, and it includes a rechargeable battery set, a power meter, and an auto-recharge device. The power meter is utilized for supervising the current power volume of the rechargeable battery set, and indicates the user how long the head-mounted bio-signal acquisition device 100 is used. Moreover, when the rechargeable battery set is running out of power, the auto-recharge device is utilized for recharging the head-mounted bio-signal acquisition device 100 through USB device or DC adapter. In the present embodiment, the power management module 140 contains a standby mode, which will be activated when the head-mounted bio-signal acquisition device 100 shut down or the biomedical probe sensor 111 did not receive any normal signal, so as to save the power and create more durability.

Next, the operation of the signal analysis processor 200 is detailed as follows. The signal analysis processor 200 includes a wireless communication module 230 as well, which receives the digital bio-signal from the head-mounted bio-signal acquisition device 100 through the communication protocol, such as WiFi, WiMax, Zigbee, RF, optical coupler, Bluetooth, or GPRS.

The signal analysis processor 200 further includes a signal process platform 210. In one embodiment, the signal process platform 210 is a cell phone, a PDA, or a portable device. In this embodiment, the signal process platform 210 is adapted and carried easily in any environment, such as the dwelling house, the hospital, or the car. However, the signal process platform 210 is not limited to the above definition. For example, the signal process platform 210 is a data processing server, such as a computer serve, a workstation, a desktop, or a laptop, setting up in the house or the hospital for analyzing data and supervising the bio-signal condition. That is, in other embodiment, the signal process platform 210 is assigned by different conditions depending on design requirement.

After receiving the digital bio-signal from the head-mounted bio-signal acquisition device 100, the signal process platform 210 will analyze the bio-signal and sent the results to the environment control module 220. The environment control module will output a control signal, based on the analysis result, to the environment control equipment 300 in order to control or adjust the environment condition, such as temperature, humidity, brightness, or sound volume. Here, the environment control equipment 300 includes a warning equipment, a air-condition equipment, and/or an electrical equipment, such as a heater, a cooler, an exsiccator, an audio system or a lighting device. Furthermore, the environment control module 220 delivers the corresponding sound or light warning signal based on the analysis result to warn the user or supervisor. Or the environment control module 220 also sends out a corresponding health index signal, which may include Electro-encephalogram (EEG), Electro-oculography (EOG), or Electro-myogram (EMG) result.

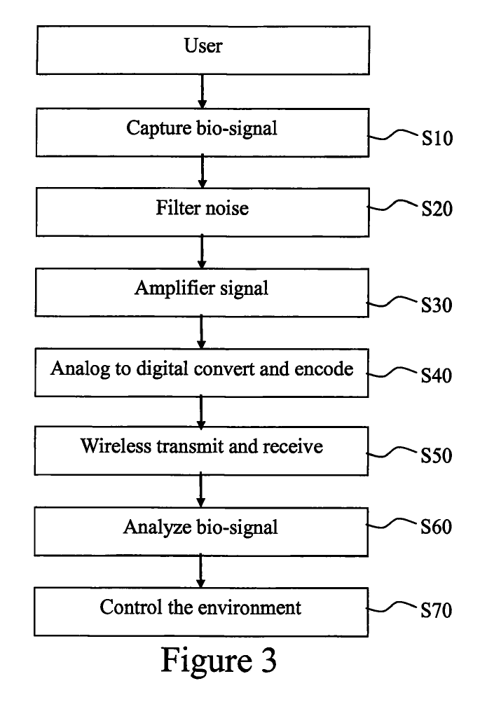

FIG. 3 is a flowchart showing an embodiment of operations of the automatic bio-signal supervising system. The present embodiment measures the bio-signal of the head according to the determinable signal range shown in Chart 1. As shown in Step 10, the bio-signals acquired from the user's forehead area and filtered the noise by the signal filters (Step 20) and then amplified by the amplifier (Step 30). In Step 40, the analog-to-digital converter (ADC) will convert the analog bio-signal into the digital bio-signal. And the digital bio-signal is encoded by the encoder. As shown in Step 50, the encoded signal is sent to the head-mounted signal process platform through the wireless communication module. The head-mounted signal process platform will perform real-time bio-signal analysis, which includes (1) extracting the desired bio-signal, (2) transferring the frequency spectrum signal to analyze and determine the current spectrum, and (3) analyzing the user's condition based on the algorithms for the drowsiness detection, the distraction evaluation, or carsick degree test (Step 60). Finally, as shown in Step 70, the automatic bio-signal supervising system delivers a control signal, a biofeedback signal, or a health index signal to control environment properly.

The present invention is applied to various environments, such as in a dwelling house, in a hospital treatment room, or in the car. Taking the drowsiness detection for instance, when the elder is taking a nap in the dwelling house or in a hospital treatment room, the automatic bio-signal supervising system in the present invention detects the drowsiness situation, and then controls the environment equipment to deliver a better and comfortable environment for sleeping. For example, the automatic bio-signal supervising system controls the air-conditioner to adjust the environmental temperature, controls the lamp to adjust the indoor lightness, or controls other electrical devices. Additionally, the automatic bio-signal supervising system in the present invention is utilized in safety auxiliary. For example, the automatic bio-signal supervising system detects the user's distraction or drowsiness condition, and then provides a warning signal to activate the ring of cell phone or the music in the audio system, so as to regain the concentration of the user.

Those described above are only the preferred embodiments to exemplify the present invention but not to limit the scope of the present invention. Any equivalent modification or variation according to the shapes, structures, features and spirit disclosed in the specification is to be also included within the scope of the present invention.

What is claimed is:

1. An automatic bio-signal supervising system for medical care, comprising:
a head-mounted bio-signal acquisition device, wore upon a head of a user, comprising a plurality of bio-signal acquisition modules, for acquiring a plurality of bio-signals from said head of said user, and converting said bio-signals into a plurality of digital bio-signals; and
a signal analysis processor for receiving said digital bio-signals acquired by said bio-signal acquisition modules, analyzing said digital bio-signals, and generating a control signal to at least a environment control equipment for controlling the environment condition of said user.

2. The automatic bio-signal supervising system for medical care of claim 1, wherein said head-mounted bio-signal acquisition device is in a stripe shape, and said bio-signal acquisition modules are distributed setup and surrounded said head of said user.

3. The automatic bio-signal supervising system for medical care of claim 1, wherein said bio-signal acquisition module comprises a biomedical probe sensor, a filter, an bio-signal amplifier, and an analog-to-digital converter (ADC), wherein said biomedical probe sensor is utilized for detecting said analog bio-signals of said user, said filter is utilized for filtering noise of analog said bio-signals, said bio-signal amplifier is utilized for amplifying said analog bio-signals, and said analog-to-digital converter (ADC) is utilized for converting said analog bio-signals into said digital bio-signals.

4. The automatic bio-signal supervising system for medical care of claim 1, wherein said head-mounted bio-signal acquisition device further comprises a signal encoding module for encoding said digital bio-signals.

5. The automatic bio-signal supervising system for medical care of claim 1, wherein said head-mounted bio-signal acquisition device further comprises a wireless communication module for wireless transmitting said digital signal to said signal analysis processor.

6. The automatic bio-signal supervising system for medical care of claim 5, wherein said wireless communication module is embodied by WiFi, WiMax, Zigbee, RF, optical coupler, Bluetooth or GPRS communication protocols.

7. The automatic bio-signal supervising system for medical care of claim 1, wherein said head-mounted bio-signal acquisition device further comprises a power management module for providing power.

8. The automatic bio-signal supervising system for medical care of claim 7, wherein said power management module comprises a rechargeable battery, a power meter, and a auto-charge device, wherein said power meter is utilized for supervising the power condition of said rechargeable battery, and said auto-charge device is utilized for activating auto-charging while said rechargeable battery is running out of power, and said power management module comprises a standby mode for saving the power of said rechargeable battery.

9. The automatic bio-signal supervising system for medical care of claim 1, wherein said signal analysis processor is a cell phone, a PDA, a mounted electrical device, or a head-mounted signal process platform.

10. The automatic bio-signal supervising system for medical care of claim 1, wherein said signal analysis processor is a server, a workstation, a desktop, a laptop, or a database server.

11. The automatic bio-signal supervising system for medical care of claim 1, wherein said signal analysis processor comprises an environment control module for providing said control signal to said environment control equipment based on the analysis result of said digital bio-signals.

12. The automatic bio-signal supervising system for medical care of claim 1, wherein said environment control equipment comprises a warning device, an air-conditioner, or an electrical device.

13. The automatic bio-signal supervising system for medical care of claim 12, wherein said environment control equipment comprises a heater, a cooler, an exsiccator, an audio system, or a lighting device.

14. The automatic bio-signal supervising system for medical care of claim 1, wherein said signal analysis processor generates a corresponding warning signal based on the analysis result of said digital bio-signal, and said warning signal comprises sound or lightness.

15. The automatic bio-signal supervising system for medical care of claim 1, wherein said signal analysis processor generates a corresponding health index signal based on the analysis result of said digital bio-signal, and said health index signal comprises the detecting result of user's Electro-encephalogram (EEG), Electro-oculography (EOG), or Electro-myogram (EMG).

16. The automatic bio-signal supervising system for medical care of claim 1, wherein said signal analysis processor comprises a wireless communication module for wireless receiving said digital bio-signal from said head-mounted bio-signal acquisition device.

17. The automatic bio-signal supervising system for medical care of claim 16, wherein said wireless communication module is embodied by WiFi, WiMax, Zigbee, RF, optical coupler, Bluetooth or GPRS communication protocols.
